(19) United States
(12) Patent Application Publication
(10) Pub. No.: US 2011/0274746 A1
(43) Pub. Date: Nov. 10, 2011

(54) Therapeutic Liposomes and Methods For Producing and Using the Same

(76) Inventors: Michael A. Schmidt, Boulder, CO (US); Joseph J. Miller, Saint Charles, IL (US)

(21) Appl. No.: 13/104,928

(22) Filed: May 10, 2011

Related U.S. Application Data
(60) Provisional application No. 61/425,366, filed on Dec. 21, 2010.
(60) Provisional application No. 61/333,173, filed on May 10, 2010.

(51) Int. Cl. A61K 9/127; A61K 31/155; A61K 31/05; A61K 31/12; A61K 31/56; A61P 9/00; A61K 31/716; A61K 31/202; A61P 3/00; A61P 31/00; A61P 29/00; A61K 31/445; A61K 31/7008

(52) U.S. Cl. 424/450; 514/317; 514/634; 514/733; 514/679; 514/169; 514/62; 514/54; 514/560

(57) ABSTRACT

The present invention provides therapeutic liposomes and methods for producing and using the same. In particular, therapeutic liposomes of the invention comprise phospholipids comprising C₁₆-C₂₂ fatty acid ester moieties. In some embodiments, these therapeutic liposomes are used in assisting delivery of an active compound, e.g., a drug and/or a nutraceutical, to a subject. In some embodiments, compositions of the invention have synergistic therapeutic effect.

### CROSS-REFERENCE TO RELATED APPLICATIONS

This application claims the priority benefit of U.S. Provisional Application No. 61/425,366, filed Dec. 21, 2010, which is related to U.S. Provisional Application No. 61/333,173, filed May 10, 2010, all of which are incorporated herein by reference in their entirety.

### FIELD OF THE INVENTION

The present invention relates to therapeutic liposomes and methods for producing and using the same. In particular, therapeutic liposomes of the invention comprise phospholipids comprising C₁₆-C₂₂ fatty acid ester moieties. In some embodiments, these therapeutic liposomes are used in assisting delivery of an active compound, e.g., drug and/or nutraceutical, to a subject. In some embodiments, such compositions have synergistic therapeutic effect.

### BACKGROUND OF THE INVENTION

Liposomes are artificially prepared vesicles made up of one or more lipid bilayers. Typical lipid bilayer of liposomes are comprised of phospholipids. Often liposomes are used for targeted drug or nutrient delivery due to their unique properties. A liposome encapsulates a region on aqueous solution inside a hydrophobic membrane. Dissolved hydrophilic solutes cannot readily pass through the lipids. However hydrophobic chemicals can be dissolved into the membrane. Thus, liposomes can carry both hydrophobic and hydrophilic molecules.

Liposomes are typically formed by combining phospholipids and the desired active compound (e.g., drugs, nutrients, nutraceuticals, etc.) in an aqueous solution. Typically, energy is supplied to the aqueous solution to form lipid bilayers that encapsulate the active compound. In some instances, the amount and type of energy supplied affects the formation and size of the liposomes, for example, often higher the energy supplied, the smaller the liposomes.

To deliver the active compound to the sites of action, the lipid bilayer from the liposome fuses or binds with cell membrane bilayers. This allows the active compounds to be indiscriminately delivered past the lipid bilayer of the cell membrane. Another method of delivery is by diffusion and not direct cell fusion. Here, the active compounds become neutralized because of the pH imbalance within the liposome, thereby allowing the active compounds to freely pass through the cell membrane.

Zerouga et al. discloses a phospholipid having a covalently attached docosahexaenoic acid (DHA) ester and methotrexate (MTX). Anticancer Drug, 2002, 13 (3), 301-311. This phospholipid is amenable to a liposome bilayer formation. However, the active compound, MTX, is not encapsulated by the liposome, but rather is part of the phospholipid itself, thereby leaving the active compound, MTX, exposed to the body's immune system.

Liposomes are becoming increasingly valuable as targeted therapy, biologics, gene therapy, and individualized medicine become more prevalent. The problem with current liposomes is that the phospholipids may not be compatible with the active compound being delivered, resulting in deactivation. Also, the phospholipids may not be compatible with the target cell. Another disadvantage with liposomes is that the immune system easily recognizes foreign substances as a threat. Thus, liposomes created from phospholipids that are easily recognized as foreign (i.e., antigen) by the immune system may be destroyed and never reach the intended target cell. Furthermore, conventional liposomes made from various phospholipids are in and of themselves non-active. That is they have shown no significant therapeutic activity besides the active compound that is encapsulated within the liposome. It is believed that if the liposome itself is therapeutic or if it can provide synergistic therapeutic activity in combination with an active compound, the amount of active compound needed can be reduced thereby significantly reducing the occurrence and/or severity of any side-effects produced by the active compound.

Therefore, there is a need for therapeutic liposomes or liposomes that enhance the activity of an active compound, e.g., liposomes that show synergistic effect when combined with an active compound.

### SUMMARY OF THE INVENTION

Some aspects of the invention provide therapeutic liposomes and therapeutic liposome compositions comprising phospholipids that can be tailored to a specific treatment to deliver active compound. In other aspects, methods are provided for encapsulating an active compound to form a therapeutic liposome composition. Such therapeutic liposome compositions can be used to reach and deliver the active compound to a target cell or organ with a minimal immune response, if at all, from the subject's immune system. Still other aspects of the invention provide therapeutic liposomes and therapeutic liposome compositions comprising a relatively high binding affinity to certain proteins located in malignant and/or benign tumors. In some particular embodiments, compositions and methods of the invention are useful as drug delivery system. Often such compositions and methods allow more active compound to be delivered to the target cell in a single dose, and/or less active compound to be delivered to healthy cells and organs, i.e., undesired sites. This is particularly useful in cancer therapy, where the active compound is usually toxic to all cells and/or organs including healthy cells and/or organs. Furthermore, in some embodiments such compositions show synergistic effect between the therapeutic liposome and the active compound.

One particular aspect of the invention provides therapeutic liposomes, therapeutic liposome compositions, and methods for using and producing the same. Typically, such therapeutic liposomes comprise a phospholipid that comprises at least two fatty acid ester moieties each of which is derived independently from a C₁₆-C₂₂ fatty acid. In some embodiments, each of the C₁₆-C₂₂ fatty acid is independently an omega-3 fatty acid, an omega-6 fatty acid, or an omega-9 fatty acid. Exemplary omega-3 fatty acids include, but are not limited to, docosahexaenoic acid (DHA), eicosapentaenoic acid (EPA), stearidonic acid (SDA), α-linolenic acid (ALA), all-cis 8,11,14,17-eicosatetraenoic acid (ETA or ETA-3, which is used interchangeably herein), and docosapentaenoic acid (DPA). Exemplary omega-6 fatty acids include, but are not limited to, γ-linolenic acid (GLA), dihomo-γ-linolenic acid (DGLA), calendic acid (CLA), and docosapentaenoic acid (DPA).

Still in other embodiments, the phospholipid further comprises choline, serine, inositol, ethanolamine, or another polar group. The phospholipids can be derived from yeast, marine animal (such as krill), plant, and/or marine plant (such as microalgae). In the case of microalgae, the biomass containing the phospholipids can be grown under specific conditions designed to alter their structure, thereby producing phospholipids with structured (i.e., non-native) phospholipids, defined as being structured differently (i.e., with different fatty acids than if grown in their native form). The phospholipids can also be synthetically modified (e.g., catalytically “re-randomized”), e.g., via esterfication/transesterfication/acylation. Such synthetic modifications (e.g., re-randomization) can be used to prepare phospholipids comprising a wide variety of different fatty acid esters and/or a wide variety of combination of fatty acid esters. Furthermore, such synthetic modification also provides a wide variety of polar groups to be attached to the phosphate moiety of the phospholipid. Such synthetic modification can be achieved using an enzyme, a chemical catalyst, ultrasound, electromagnetic energy, or a combination thereof. In some embodiments, synthetic modification comprises reacting phospholipids with fatty acids, to modify (e.g., rearrange) one or more of the terminal positions associated with the phospholipid, removing or hydrolyzing at least one fatty acid ester group to produce the hydroxyl group, and covalently attaching (i.e., forming an ester group with) at least one specific fatty acid of C₁₆ or higher, e.g. C₁₆, C₁₈, C₂₀, or C₂₂, to produce the fatty acid ester group.

Yet other aspects of the invention provide therapeutic liposomes comprising a phospholipid that comprises at least two ester moiety of C₁₆-C₂₂ fatty acids. It should be appreciated that unlike conventional liposomes made from phospholipids, liposomes of the present invention in and of themselves are therapeutic (e.g., shows therapeutic activity). In many instances, liposomes of the invention also show synergistic effect when combined with an active compound. The fatty acid moieties can be saturated or unsaturated omega-3, omega-6, and/or omega-9 fatty acids. In some embodiments, the phosphate moiety of the phospholipid comprises a phosphoester moiety that comprises a polar functional group on its terminal position. Exemplary phosphoester moieties include, but are not limited to, a phosphoester of choline, serine, inositol, and ethanolamine. In other embodiments, the phospholipids are capable of substantially encapsulating an active compound. Still in other embodiments, the fatty acids are substantially non-immunogenic or adapted to minimize detection by a living organism, such as the human body.

Still other aspects of the invention provide therapeutic liposome compositions comprising a therapeutic liposome and an active compound that is encapsulated within the therapeutic liposome. In some embodiments, each of the fatty acid moiety in the phospholipid is independently EPA, SDA, DPA, ETA (i.e., ETA-3), or DHA.

Yet in other embodiments, the therapeutic liposome composition comprises a mixture of different phospholipids. In one particular embodiment, the liposome comprises a mixture of a phospholipid comprising phosphate ester of choline and two DHA esters; a second phospholipid comprising phosphate ester of choline and DHA and EPA esters; a third phospholipid comprising phosphate ester of choline and DHA and ETA esters; and a fourth phospholipid comprising phosphate ester of choline and two ETA esters. Within these embodiments, the first through fourth phospholipids can be present in an amount ranging from about 5%-95% each by weight as long as the total amount of the phospholipids does not exceed 100%. The phospholipids can also be present in equal weight amounts.

Still other aspects of the invention provide therapeutic liposome compositions that comprise a therapeutic liposome disclosed herein and an active compound that is encapsulated substantially within the therapeutic liposome. The active compound can be virtually any molecule such as drugs, nutraceuticals, nutrients, etc. Some examples of active compound include, but are not limited to, organic acids, lipids, carotenoids, amino acids, nucleotides, steroids, trace elements, vitamins, hormones, peptides, amino acids, proteins, polyphenols, quinones, combinations of molecules, drugs, polymeric compounds (such as glycogen derived molecules, or glycosaminoglycans, or other polymeric units that form tissue such as bone and cartilage), as well as a combination thereof.

In some embodiments, the active compound can comprise a drug selected from the group consisting of an antibiotic, a statin, a calcium channel blocker, an angiotensin-converting enzyme (ACE) inhibitors, an α-agonist, an α-blocker, a rennin blocker, dexmethylphenidate, guanfacine, methylphenidate, and an anti-inflammatory.

Other aspects of the invention provide methods for making therapeutic liposome compositions disclosed herein. In some embodiments, such methods include admixing or combining a solubilized phospholipid mixture with a buffer solution comprising an active compound; and forming a liposome encapsulated active compound composition. The solubilized phospholipid mixture comprises a solubilized phospholipid in a solvent. The active compound is substantially encapsulate within the therapeutic phospholipid liposome. In some embodiments, methods of the invention can include removing at least a portion of the solvent from the therapeutic liposome composition.

One particular aspect of the invention provides a therapeutic liposome composition comprising a phospholipid that comprises at least two fatty acid ester moieties each of which is independently a C₁₆-C₂₂ fatty acid; and an active compound.

Within this aspect of the invention, in some embodiments, the phospholipid is of the formula:

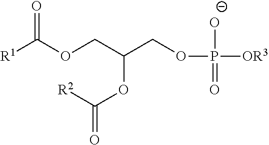

where
each of R¹ and R² together with the carboxylate group to which they are attached to is independently a C₁₆-C₂₂ fatty acid moiety; and
R³ together with the oxygen atom to which it is attached forms choline, serine, inositol, or ethanolamine moiety.

In another embodiment, the C₁₆-C₂₂ fatty acid is an omega-3 fatty acid, an omega-6 fatty acid, or an omega-9 fatty acid.

Still in another embodiment, the C₁₆-C₂₂ fatty acid comprises α-linolenic acid (ALA), docosahexaenoic acid (DHA), eicosatetraenoic acid (ETA or ETA-3), eicosapentaenoic acid (EPA), stearidonic acid (SDA), γ-linolenic acid (GLA), calendic acid (CLA), docosapentaenoic acid (DPA), or a combination thereof.

Yet in other embodiments, each of the C₁₆-C₂₂ fatty acid is independently SDA, DPA, DHA, EPA, or ETA.

In other embodiments, the active compound comprises a drug or a nutraceutical compound. Within these embodiments, in some instances, the drug comprises an antibiotic, a statin, a calcium channel blocker, an ACE inhibitor, an α-agonist, an α-blocker, a rennin blocker, dexmethylphenidate, guanfacine, methylphenidate, an anti-inflammatory compound, or a combination thereof. In other instances, the nutraceutical compound comprises resveratrol, a quinone, diferuloylmethane, a sterol, a β-glucan, glucosamine, a carotenoid, a terpene, a xanthpohyll, an omega-3 fatty acid, a probiotic, a prebiotic, or a combination thereof.

Another particular aspect of the invention provides a method for making a therapeutic liposome composition comprising a therapeutic liposome and an active compound that is encapsulated within the therapeutic liposome. Typically, the therapeutic liposome comprises a phospholipid that comprises at least two fatty acid ester moieties each of which is independently a C₁₆-C₂₂ fatty acid, said method comprising:
combining a solubilized phospholipid mixture with a buffer solution comprising an active compound, wherein the solubilized phospholipid mixture comprises a solubilized phospholipid in a solvent; and
forming a therapeutic liposome composition comprising active compound that is encapsulated within therapeutic liposome.

In some embodiments, the step of forming the therapeutic liposome composition comprises sonication, homogenization, microfluidization, laser, high-shear mixing, or a combination thereof.

In other embodiments, such methods further comprise the step of removing at least a portion of the solvent from the therapeutic liposome composition.

Still in other embodiments, the solvent comprises hexane, isohexane, chloroform, polysorbate, a sugar-alcohol compound; Vitamin-E TPGS; polyethylene glycol, a petrochemical, or a combination thereof.

In some particular embodiments, the phospholipid is of the formula:

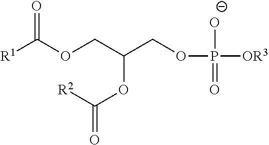

where
each of R¹ and R² together with the carboxylate group to which they are attached to is independently a C₁₆-C₂₂ fatty acid moiety; and
R³ together with the oxygen atom to which it is attached forms choline, serine, inositol, or ethanolamine moiety.

Yet in other embodiments, each of the C₁₆-C₂₂ fatty acid is independently an omega-3 fatty acid, an omega-6 fatty acid, or an omega-9 fatty acid. Within these embodiments, in some instances, each of the C₁₆-C₂₂ fatty acid is independently α-linolenic acid (ALA), docosahexaenoic acid (DHA), eicosatetraenoic acid (ETA or ETA-3), eicosapentaenoic acid (EPA), stearidonic acid (SDA), γ-linolenic acid (GLA), calendic acid (CLA), or docosapentaenoic acid (DPA).

Still in other embodiments, each of the C₁₆-C₂₂ fatty acid is SDA, DPA, DHA, EPA, or ETA.

In other embodiments, the active compound comprises a drug or a nutraceutical compound. Within these embodiments, in some instances the drug comprises an antibiotic, a statin, a calcium channel blocker, an ACE inhibitor, an α-agonist, an α-blocker, a rennin blocker, dexmethylphenidate, guanfacine, methylphenidate, an anti-inflammatory compound, or a combination thereof. In other instances, the nutraceutical compound comprises resveratrol, a quinone, diferuloylmethane, a sterol, a β-glucan, glucosamine, a carotenoid, a terpene, a xanthpohyll, an omega-3 fatty acid, a probiotic, a prebiotic, or a combination thereof.

Therapeutic liposome compositions of the invention can also be a “single-shelled”, “double-shelled” or a combination thereof. Single-shelled (or unilamellar) refers to a liposome comprising a single lipid bilayer. Double-shelled (or multilamellar) refers to a liposome comprising two lipid bilayer, e.g., one lipid bilayer liposome encapsulated within another lipid bilayer liposome.

### DETAILED DESCRIPTION OF THE INVENTION

Some aspects of the invention provide therapeutic liposomes comprising a phospholipid that comprises at least two attached fatty acids. Each of the fatty acid is independently a C₁₆-C₂₂ fatty acid. In addition, each of the fatty acid can be saturated or unsaturated. In some embodiments, the fatty acid is unsaturated. Within these embodiments, in some instances the fatty acid is an omega-3, an omega-6, or omega-9 fatty acid. The phospholipid also comprises a phosphate ester. Typically, phosphate ester comprises a polar compound that is attached to the phosphate group (thus forming a phosphate ester group). In some embodiments, the polar compound comprises a polar group such as an amino group, mono-, di-, tri-, or tetralkyl-amino, group, hydroxyl group, thiol group, etc. Exemplary polar compounds that can be attached to phosphate group of the phospholipid include, but are not limited to, choline, serine, inositol, and ethanolamine.

One particular embodiment of the phospholipids of the invention is shown below as FIG. 1. In FIG. 1, moieties A and B are derived independently from a C₁₆-C₂₂ fatty acid and moiety C is derived from a polar compound.

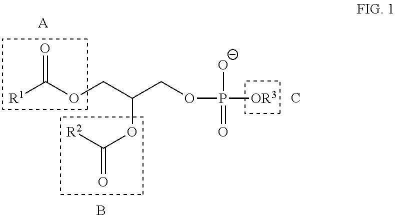

It should be appreciated that the phospholipid shown in FIG. 1 has at least one chiral center (e.g., the carbon atom to which moiety B is attached). Accordingly, there can be at least two stereoisomer within the phospholipid shown in FIG. 1. The scope of the invention includes all isomers and mixture of isomers of phospholipids. In some embodiments, liposomes of the invention comprise an enantiomerically enriched or enantiomerically pure phospholipid. In other embodiments, liposomes of the invention comprise a racemic mixture of phospholipid of FIG. 1.

The phospholipids that form the therapeutic liposome do not have to all be the same. In some embodiments, varying the amount of different phospholipids in producing a therapeutic liposome can increase the therapeutic effectiveness (e.g., delivery to a target cell or organ) of the liposome. In many instances, there is a synergistic effect between the therapeutic liposome and the active compound which is encapsulated within the liposome. Various combinations of fatty acids, and polar compounds can be used forming a therapeutic liposome. For example, in some embodiments 5%-95% by weight, including 5%, 15%, 25%, 35%, 45%, 55%, 65%, 75%, 85%, and 95%, of the therapeutic liposome can comprise phospholipids that comprise DHA, ETA, and choline; while the remaining phospholipids can comprise EPA, DHA, and serine. Yet in other embodiments, 5%-95% by weight, including 5%, 15%, 25%, 35%, 45%, 55%, 65%, 75%, 85%, and 95%, of the therapeutic liposome can comprise phospholipids having a same fatty acid (e.g., comprising ETA or ETA-3) and choline as the polar compound, while the remaining phospholipids can comprise DHA and ETA, and serine. Further, the therapeutic liposomes of the invention are not limited to one or two different phospholipids, but rather can be composed of 3 to 100 different phospholipids, including 5-80, 10-70, 20-60, 30-50, and about 40.

The composition of any one fatty acid in the unloaded (i.e., without any encapsulated active compound) therapeutic liposome can vary from about 5% to about 95% by weight of the total liposome including about 5%, 10%, 15%, 20%, 25%, 30%, 35%, 40%, 45%, 50%, 55%, 60%, 65%, 70%, 75%, 80%, 85%, 90%, and 95% by weight of the total liposome. For example, a therapeutic liposome can comprise about 30% by weight DHA, about 30% by weight EPA, and about 30% by weight ETA, with the remainder of the weight typically being the phosphate ester group and the diglyceride back bone. Alternatively, about 50% by weight of the therapeutic liposome can comprise DHA and about 20% by weight of the therapeutic liposome can comprise ETA. Further, the therapeutic liposomes are not limited to two or three different fatty acids, but rather can compose several different fatty acids, including 4, 5, 6, 7, 8, 9, and 10. These additional fatty acids can include DPA, ALA, GLA, CLA, ETA, EPA, DHA, SDA, or a combination thereof. Also, the therapeutic liposome can comprise various combinations of omega-3s, omega-6s, and omega-9s, so long as the total concentration of fatty acids does not exceed about 95% by weight.

Similarly, the composition of any one type of polar compound (i.e., moiety C in FIG. 1) can vary from about 5% to about 45% by weight of the unloaded therapeutic liposome, including about 5%, 10%, 15%, 20%, 25%, 30%, 35%, 40%, and 45% by weight of the unloaded therapeutic liposome. For example the liposome can comprise about 15% by weight choline, about 15% by weight serine, and about 10% by weight inositol. Alternatively, the therapeutic liposome of the invention can comprise about 20% by weight choline and about 15% by weight serine, or 30% by weight choline and the remainder a mixture of various polar compounds. Further the therapeutic liposomes are not limited to just 1-3 polar groups, but can comprise several different types, including 4 and 5, so long as the total concentration of polar groups does not exceed about 45% by weight.

The type and quantity of fatty acid and polar compound typically depend on the phospholipid source. For example, plant based phospholipid sources, such as palm, echium, and soy, may be high in choline moiety, but low in certain omega-3 fatty acids. Conversely, marine microorganisms, such as hill and fish may be high in both serine polar moiety and certain omega-3 fatty acids, such as EPA.

Microbial phospholipids, such as marine plants, plankton, fungi, microalgae, and macroalgae comprise a wide range of omega-3, omega-6, and omega-9 fatty acids and polar groups, depending on the source. For phospholipids comprising DHA, the microalgae can include species from the genera Thraustochytrium, Schizochytrium, and Crypthecodinium, including Crypthecodinium cohnii (C. cohnii). Also, members of the class Dinophyceae, Bacillariophyceae, Chlorophyceae, Prymnesiophyceae, and Euglenophyceae can produce suitable phospholipids with high concentrations of DHA. For phospholipids containing EPA, the microalgae can include species from the genera Thraustochytrium Schizochytrium, Phaeodactylum, Nannochloropsis, Porphydrium, and Monodus, including Phaeodactylum tricomulum, Porphyridium cruentum, and Monodus subterranous.

Other microalgae that produce DHA and EPA include, but are not limited to, Odentella aurita, Pavolova lutheri, Isochysis galbana, Nannochloropsis, and Porphyridium cruentum. Still other useful microalgae include Chaetoceros calcitrans, Chaeotoceros gracilis, Nitzichia cloesterium, Skeletonema costatum, Thalassiosira pseudonana, Dunaliella tertiolecta, Nannochloris atomus, Chroomonas salina, Nannochloropsis oculata, Tetraselmis chui, Tetraselmis suecica, and Pavlova salina.

Cultivation techniques of the above microalgae are well known. For example, cultivation of Thraustochytrium and Schizochytrium is disclosed in U.S. Pat. No. 5,340,594, and cultivation of C. cohnii is disclosed in U.S. Pat. Nos. 5,397,591 and 5,492,538 and Japanese Patent Publication (to Kokai) No. 1-199588 (1989).

In some embodiments, phospholipids are isolated and purified from the above marine biomasses. Impurities, such as bacteria, particulates, and extraction chemicals, are almost always present when the phospholipids are extracted. Extraction of the phospholipids can be done using known methods, including polar and non-polar solvent extraction, spray drying, super critical extraction, centrifuge, enzymatic extraction, mechanical press, extrusion, sonication, decanter extraction, and combinations thereof.

One method of extracting phospholipids is to spray dry the marine biomass, which typically lyses the cells. The lysed cells are then extracted typically using a non-polar solvent, such as hexane, to remove the fatty acid phospholipid portion. Such process, and other suitable processes, is described in detail in U.S. Pat. No. 6,372,460. Another method is to pretreat the biomass to deactivate any phospholipase that may be present and would otherwise degrade the phospholipids. The lipids and phospholipids are then extracted from the biomass using a known technique, including polar and non-polar solvent extraction, spray drying, super critical extraction, centrifuge, enzymatic extraction, mechanical press, extrusion, and decanter extraction. The extracted phospholipids are often isolated and purified from the total lipid fraction with water wash, acetone, or other solvents that cause separation of the hydrophobic or neutral materials from polar and glycolipids. The phospholipids are then dried using a known method such as wiped-film evaporation.

Any bacteria present in the mircobiomass or phospholipid can be inactivated using an anti-bacterial agent. Particulates can be removed, e.g., by filtration using any of the various filtration methods known to one skilled in the art, such as centrifuge, filter press, cyclone filtration, gravity decanter, or filter media. Extraction solvenats can be removed using flash distillation, evaporation, and gravity decanting.

C. cohnii contains high concentrations of phospholipids (e.g., 20%-30%) with a high amount of DHA (e.g., 25%-45%). The phospholipids can be further purified and isolated to concentrations of around 90%. Species from the genera Phaeodactylum, Nannochloropsis, Porphydrium, and Monodus, also contain high concentrations of phospholipids with a high amount of EPA. As with C. cohnii, the phospholipids from these microorganisms can be further purified and isolated to concentrations of around 90%.

Further purification of the phospholipids allows production of therapeutic liposomes with a desired amount of fatty acid concentrations. For example, the therapeutic liposome can contain about 40% DHA and about 40% EPA (present as 90% by weight phospholipids), as disclosed above, or 70% EPA and 10% DHA, or 10% EPA and 70% DHA. Further, DPA, SDA, GLA, ALA, ETA, CLA, or a combination thereof fatty acid phospholipids can be incorporated into the therapeutic liposome by isolating and purifying the phospholipids comprising these fatty acids.

Some aspects of the invention provide therapeutic liposomes comprising a mixture of phospholipids. In some embodiments, the therapeutic liposome comprises a mixture of phospholipids. In one particular embodiment, a mixture of phospholipids comprises phospholipids comprising a various amounts of EPA, ETA, and DHA. For example, one phospholipid can have EPA and DHA fatty acid ester moiety, while a second phospholipid can have ETA and DHA fatty acid ester moiety. It should be appreciated that the therapeutic liposome can have two or more, three or more, four or more, or five or more different phospholipids. In addition, fatty acids of the therapeutic liposome can include SDA, DPA, EPA, ETA-3, DHA, and a combination thereof.

In some embodiments, the concentration of each of DHA, ETA, and EPA in the therapeutic liposome can vary from less than about 10% to about 95% by weight total fatty acids, including about 5%, 10%, 15%, 20%, 25%, 30%, 35%, 40%, 45%, 50%, 55%, 60%, 65%, 70%, 75%, 80%, 85%, 90%, and 95%. Other fatty acids, such as DPA, ALA, GLA, CLA, SDA, and SDA can also be present in the therapeutic liposome. The concentration of these other fatty acids is typically less than the concentration of DHA, ETA, and EPA. The total omega-3, omega-6, and/or omega-9 fatty acid content in the therapeutic liposome is typically greater than about 15% to about 70%, including about 15%, 20%, 25%, 30%, 35%, 40%, 45%, 50%, 55%, 60%, 65%, and 70% by weight.

Some of the typical lipid source include plant or marine organisms, which contain choline or serine. Exemplary plant lipid sources include, but are not limited to, soy lecithin, borage oil, primrose oil, sunflower oil, palm oil, echium, and combinations thereof. Exemplary marine organism lipid sources include, but are not limited to, hill and fish. Yeast can also be the lipid source. Thus, lipids can be derived from microbial phospholipids, fish oil, krill oil, plant oil, yeast, and a combination thereof. Lipids can be saturated or unsaturated fatty acids in the form of free fatty acids, fatty acid chlorides, fatty acid alkyl esters, fatty acid vinyl esters, fatty acid anhydrides, mono, di, or tri-glycerides, or any other activated form of fatty acids. When a microbial phospholipid is used, the phospholipid can be isolated and purified from a marine biomass derived from plankton, fungi, macroalgae, and/or microalgae. Suitable microalgae can include species from the genera Thraustochytrium, Schizochytrium, and Crypthecodinium, including Crypthecodinium cohnii (C. cohnii), Dinophyceae, Bacillariophyceae, Chlorophyceae, Prymnesiophyceae, Euglenophyceae, Phaeodactylum, Nannochloropsis, Porphydrium, and Monodus, including Phaeodactylum tricomulum, Porphyridium cruentum, and Monodus subterranous.

The total concentration of the phospholipids in the therapeutic liposome can vary from about 5% to about 95% by weight of the liposome, including about 5%, 10%, 15%, 20%, 30%, 35%, 40%, 45%, 50%, 55%, 60%, 65%, 70%, 75%, 80%, 85%, 90%, and 95% by weight of the therapeutic liposome.

The fatty acids on the phospholipid can be present in a purely random fashion, i.e., some phospholipids may have all C₂₀ or C₂₂ fatty acids, a mixture of both, or a mixture of C₂₀, C₂₂, C₁₆, and C₁₈ fatty acids, depending on the concentration of fatty acid type that is desired. Thus, there is no placement predetermination of the fatty acid moiety on the phospholipids.

Further purification of the phospholipids can be used to produce phospholipids having a desired or tailored amount of fatty acids. For example, the phospholipid can contain about 40% DHA and about 40% EPA (present as 90% by weight phospholipids), or 70% EPA and 10% DHA; or 10% EPA and 70% DHA; or 40% ETA, 20% DHA, and 20% EPA. Further, phospholipids comprising DPA, SDA, GLA, ALA, CLA, or a combination thereof can be incorporated into the phospholipids by isolating and purifying (or synthesizing) the phospholipids containing these fatty acids. Thus, a truly tailored fatty acid concentration phospholipid can be obtained.

Other aspects of the invention provide methods for making the phospholipid composition. Such methods typically comprise providing a mixture of a first phospholipid or its source, a second lipid or its source, and a third lipid or its source under conditions sufficient to produce the desired phospholipid composition. In some embodiments, a lipase is used to produce the phospholipid composition from the mixture. In some instances, the first phospholipid comprises a fatty acid having C₂₀, C₁₈ or lower amount of carbon atoms. Still in other instances, the second lipid comprises at least one C₂₀ fatty acid, and the third lipid comprises at least one C₂₂ fatty acid. In some embodiments, methods for making the phospholipid composition comprise providing a mixture of a first phospholipid or its source and a second lipid or its source under conditions sufficient to produce the phospholipid composition. Within these embodiments, in some instances the first phospholipid comprises a fatty acid having C₂₀, C₁₈ or lower amount of carbon atoms. Still in other instances, the second lipid comprises a C₂₀ or C₂₂ fatty acid.

Synthetic modification of phospholipids typically comprises reacting the phospholipid with a fatty acid to rearrange/replace one or more of the fatty acids attached to the phospholipid or removing at least one fatty acid that is attached or covalently bonded to the phospholipid and attaching another C₁₆ or higher fatty acid, e.g., a C₁₆, C₁₈, C₂₀, or C₂₂ fatty acid. The reaction of the phospholipids or their sources with lipase-based enzyme allows modification of the fatty acids of the phospholipid. For example, at least one C₂₀, C₁₈ or lower fatty acid of the first phospholipid can be replaced with at least one C₂₀ and/or C₂₂ fatty acid from the second and/or third lipids or their sources. Thus, depending on the concentration of EPA, ETA, and DHA present in the mixture, one can produce a phospholipid comprising a desired amount of these fatty acids. For example, when an EPA or ETA phospholipid is desired, a lipid source containing C₂₀ fatty acid can be used to replace the C₁₈ or lower fatty acid from the phospholipid source in the presence of a catalyst or a lipase to produce the desired phospholipid. When a phospholipid mixture comprising DHA, EPA, and/or ETA is desired, a phospholipid or its source comprising C₂₀ fatty acid and a lipid or its source comprising C₂₂ fatty acid can be used to replace the C₂₀, C₁₈ or lower fatty acid moieties from the initial phospholipid. Thus, the original fatty acid moieties can be replaced or substituted with different fatty acid moieties (e.g., higher carbon chain fatty acids) using a catalyst or an enzyme (e.g., lipase). It should be appreciated that additional phospholipids or the corresponding sources comprising different fatty acid ester moieties can be added including lipids comprising DPA, ALA, GLA, CLA, SDA, or a mixture thereof, to replace or substitute the C₂₀, C₁₈ or lower carbon chains fatty acid moieties from the initial phospholipid.

The lipids used in a substitution reaction can be phospholipids derived from the microalgae and/or marine based phospholipids from fish or krill and/or plant based phospholipids. Alternatively, synthetic fatty acids or derivatives thereof can also be used in substitution reaction. Exemplary synthetic fatty acids or derivative that are useful in preparing a desired phospholipid composition include, but are not limited to, fatty acid chlorides, fatty acid alkyl esters, fatty acid vinyl esters, fatty acid anhydrides, mono, di, and tri-glycerides, or any other form of a fatty acid that can be used in a transesterification reaction.

Enzymes that are useful in transesterification reaction include lipase and lipase like enzyme, e.g., phospholipase enzyme. Suitable phospholipases include, but are not limited to, Phospholipase A (Lecitase) provided by Sankyo and Novozymes (435 and 525). The phospholipase can be immobilized onto an insoluble matrix and/or by coating the same with a surfactant material. For example, soy lecithin phospholipids containing phosphatidylcholine are combined in a reactor with high purity phospholipid fractions in a weight ratio of from about 25:75 to 75:25 soy lecithin phospholipid to marine phospholipid, including 50:50 weight ratio, 35:65 weight ratio, 65:35 weight ratio, 40:60 weight ratio, and 60:40 weight ratio. Lipase and/or an enzyme having lipase activity are added to the mixture in a concentration range from about 2%-5% by weight of total phospholipids.

Additionally, the polar moiety C in FIG. 1 can also be replaced or substituted with another polar compound. For example, soy lechitin phospholipid containing choline polar groups can be reacted in the presence of Phospholipase D enzyme and serine to replace all or a portion of the choline with serine. This allows for the use of cheaper plant based phospholipids with choline polar groups, rather than more expensive marine organism phospholipids with serine groups, as the starting material. The substitution on the phosphate group can be achieved simultaneously with the fatty acid substitution or such substitution can be stepwise.

The disclosed process allows for preparation of a phospholipid with tailored concentrations of ETA, EPA and/or DHA, as well as one or more of ALA, GLA, SDA, DPA,CLA, and, if desired. The advantages of a phospholipid mixture of the present invention are low cost (since the first phospholipid source can be plant based), tailored fatty acid concentrations, and high purity. The tailored fatty acid allows specific formulations to be readily manufactured (i.e., a high EPA with phosphatidylcholine (PC) or a high DHA with phosphatidylserine (PS)). The tailored phospholipids allow for the formation of unique therapeutic liposomes that can more effectively deliver active compounds to the target cell as well as, in some instances, provide synergistic effect.

The substitution reaction can be repeated several times to increase the concentration of a desired fatty acids in the phospholipid mixture. As the substitution reaches equilibrium between distribution of carbon chains amongst glycerides and phospholipids, isolation of the phospholipids from the glycerides is possible. The isolated phospholipids can be placed into new glycerides (or esters) fraction whose availability of the target carbon chain(s) is higher than that of the previous glyceride media, thus, continually reaching higher proportions of the target carbon chain(s) in the final phospholipid mixture.

A catalyst can also be used to increase the substitution rate. The catalyst can be any alkali catalyst such as a metal alkoxide (e.g., metal ethoxide and metal methoxide, where metal can be any suitable metal ion known to one skilled in the art such as, but not limited to, sodium, potassium, calcium, magnesium, strontium, etc.). Organic amines can also be used to facilitate the reaction, as well as other weakly basic compounds such as, but not limited to, sodium or potassium acetate. In some instances, sonification can also be used either by itself or in combination with the enzymes or catalysts.

The reactor for carrying out the substitution reaction can be any vessel that is suitable for holding the phopholipids or their sources including, but not limited to, a stainless steel tank, glass lined tank, and steel-alloy blend tank. The reaction temperature is typically between about 40° C. and about 100° C., including from about 50° C. to about 90° C., about 60° C. to about 80° C., and 70° C. The reaction times can range from about 4 hours to about 24 hours, including 6 hours to 20 hours, and 10 hours to 15 hours. Also, the reaction can take place at ambient temperatures, about 20° C., for about 3 months. This allows the substitution to occur during storage of the phospholipid and forgoes the need to supply additional heat.

The phospholipids can also be reacted with lipase-based compounds including those previously processed by catalysts, to hydrolyze the ester-bonds in the phospholipids. The result is a lipid or oil with a lower fish or chemical odor and an increased fruity odor. In one aspect, a hill based phospholipid is reacted with a lipase to deodorize it. The typically reaction takes place from about 40° C. to about 65° C. under vacuum and agitation for a period from about 4 to about 48 hours. The lipase is then deactivated or filtered out of the final product.

Some aspects of the invention provide a therapeutic liposome compositions comprising the phospholipid disclosed herein and an active compound that is encapsulated within the therapeutic liposome formed from the phospholipid. The active compound can be virtually any molecule such as organic acids, lipids, carotenoids, amino acids, nucleotides, steroids, trace elements, vitamins, hormones, peptides, amino acids, proteins, polyphenols, quinones, combinations of molecules, compounds such as drugs, polymeric compounds (such as glycogen derived molecules, or glycosaminoglycans, or other polymeric units that form tissue such as bone and cartilage), or one or more of these classes delivered together. The therapeutic liposome composition can be introduced into humans and animals by a variety of methods, including oral, topical, intranasal, ocular, or intravenous methods.

In some particular embodiments, the phospholipids substantially encapsulate the active compound, i.e., forms a therapeutic liposome comprising the active compound within the therapeutic liposome. The encapsulation is typically achieved by selecting a specific combination of polar groups (e.g., moiety C in FIG. 1) in relation to the active compound, so that the active compound does not diffuse through the therapeutic liposome prior to reaching the target cell. Further, the polar groups can be selected to enhance binding on a cell membrane. For example, choline polar groups are readily found in every cell in the human body. Thus, a phospholipid with a majority of choline polar groups in the lipid bilayer can easily bind onto human cells to deliver the active compound. Also, the phospholipids can be selected with specific polar groups that enhance binding affinity to certain proteins. These proteins can be associated with specific malignant or benign tumors. For example, Annexin-A5 is a naturally-occurring protein with avid binding affinity for serine polar groups, and the phospholipid in the liposome can be modified as such to be associated with malignant and benign tumors.

The phospholipid that forms the therapeutic liposome can also be selected to be synergistic with the active compound it encapsulates in achieving a combined therapeutic benefit, thus the resulting therapeutic liposome composition is therapeutic in nature rather than simply a delivery mechanism. As an example, if the desired benefit is cardiovascular, a phosphatidylcholine with EPA fatty acid esters form a therapeutic liposome to encapsulate a nutrient or drug that benefits cardiovascular health. Similarly, a phosphatidylserine with DHA fatty acid esters can be used to form a therapeutic liposome composition with a nutrient or drug acting positively in terms of cognitive health.

In one particular aspect of the disclosed therapeutic liposome composition, the active compound is an antibiotic. Such a therapeutic lipid composition allows increased selective absorption of the antibiotic at the small intestinal mucosa and minimize delivery of the antibiotic to the colonic microflora. In this aspect, the liposome encapsulated antibiotic is designed to reduce the adverse effects of antibiotics on colonic microflora, such as Clostridium difficile, Klebsiella pneumoniae, Proteus vulgaris, and others. In some instances, the therapeutic liposome encapsulated antibiotic can reduce development of antibiotic resistance in colonic microbial flora, such as Clostridium difficile, Klebsiella pneumoniae, Proteus vulgaris, and others. The antibiotics can be effective against gram negative and gram positive bacteria. Provision of the omega-3 liposomes act to maintain intestinal mucosal integrity and barrier defenses. It is believed that mitigation of delivery of the antibiotic to the colonic flora, can in some instances minimize the development of obesity, metabolic syndrome, and diabetes, and inflammatory bowel diseases, which are associated with alteration in gut microflora at the phylum, genus, and species level. Accordingly, some embodiments of the invention provide methods for reducing the risk of developing obesity, metabolic syndrome, and/or diabetes in a subject who is undergoing antibiotic treatment. Such methods include administering a therapeutic liposome composition of the invention that comprises an antibiotic as an active compound.

In one particular aspect of the disclosed therapeutic liposome composition, the active compound is an antibiotic. In some instances of this particular embodiment, the antibiotic is active in treating conditions, such as acute otitis media or chronic otitis media, conditions with a significant inflammatory component. The omega-3 enriched phospholipids that comprise the therapeutic liposome have the effect of reducing cyclooxygenase and lipoxygenase metabolites (e.g., PGE2 and LTB4) in the middle ear compartment, which acts in synergy with the antibiotic that targets microbes within the middle ear compartment. The antibiotic can be effective against gram negative and gram positive bacteria.

One particular embodiment of the therapeutic liposome composition of the invention comprises EPA and DHA phospholipids and N-acetylcysteine, as an active compound. In some instances of this particular embodiment, N-acetylcysteine is active in treating conditions, such as acute otitis media or chronic otitis media, adenoiditis, pharyngitis, pneumonia, and other conditions with a significant tendency to form bacterial biofilms. The omega-3 enriched phospholipids that comprise the therapeutic liposome have the effect of reducing the biofilm scaffolding in the middle ear, tonsil, adenoid, or other body compartment, which acts in synergy with the N-acetylcysteine that target microbial biofilms within these compartments. The therapeutic N-acetylcysteine liposome can be effective against gram negative and gram positive bacteria.

In yet another particular embodiment of the therapeutic liposome composition, a compound with therapeutic effects on intestinal mucosal integrity (including such compounds as butyrate, glutamine, glutathione, sulphasalazine, metronidazole, etc.) is encapsulated within the therapeutic liposome. In some instances of this particular embodiment, compounds like butyrate, glutamine, and glutathione are active in reducing endotoxin transit across the intestinal mucosal membrane, thereby reducing endotoxinemia. The omega-3 enriched phospholipids that comprise the therapeutic liposome have the effect of re-establishing intestinal mucosal barrier integrity, which acts in synergy with the butyrate or glutamine that is used as for repair of these membrane structures. The omega-3 enriched phospholipids containing EPA, ETA, and DHA that comprise the therapeutic liposome have a synergistic effect on barrier function in a manner that significantly increases the effect of the liposome/drug complex on intestinal barrier integrity, passage of endotoxin (LPS), and on endotoxinemia.

In yet another particular embodiment of the therapeutic liposome composition, a statin (HMG CoA Reductase Inhibitor, including but not limited to lovastatin, atorvastatin, simvastatin, etc.) is encapsulated within the therapeutic liposome. The omega-3 enriched phospholipids containing EPA that comprise the therapeutic liposome have a synergistic effect on blood lipids and lipoprotein composition (HDL, LDL, VLDL, etc) in a manner that significantly increases the effect of the liposome/drug complex on blood lipids and on the disorders associated with alterations in blood lipids.

In still a further particular embodiment of the therapeutic liposome composition of the invention, the therapeutic liposome comprises EPA-phospholipids that encapsulate drugs beneficial to cardiovascular function, such as calcium channel blockers, ACE inhibitors, α-agonists, α-blockers, renin blockers, and others. The omega-3 enriched phospholipids that comprise the therapeutic liposome have a synergistic effect on blood pressure and features associated with heart disease.

In another particular embodiment of the therapeutic liposome composition of the invention, the therapeutic liposome comprises DHA-phospholipids that encapsulate drugs beneficial to central nervous system function, such as dexmethylphenidate, guanfacine, or methylphenidate for treatment of attention deficit hyperactivity disorder (ADHD). The DHA-enriched phospholipids that comprise the therapeutic liposome have a synergistic effect with these drugs utilized to improve ADHD symptoms.

In other embodiments, the compound attached to the phosphate group can be selected such that it binds to lipid receptors, such as toll-like receptor-4 (TLR-4), which typically detect the lipopolysaccharide-A moiety of gram negative bacteria or bacterial endotoxin. Activation of the TLR-4 by endotoxin elicits an inflammatory response that can be detrimental to the organism. The liposomes with fatty acids, such as DHA, ETA, and EPA, may bind to TLR-4 without eliciting the typical inflammatory response, thus mediating disorders associated with excessive TLR-4 activation. These include inflammatory bowel diseases, disorders of excessive intestinal permeability, and conditions of Th1/Th2 immune imbalance.

Therapeutic liposomes of the invention can comprise a plurality of different phospholipids. For example, each liposome can comprise a mixture of (e.g., four different) phospholipids. In one particular embodiment, the first phospholipid comprises choline and DHA fatty acid esters; the second phospholipid comprises choline and DHA and EPA fatty acid esters; the third phospholipid comprises choline and DHA and ETA fatty acid esters; and the fourth phospholipid comprises choline and ETA fatty acid esters. The amounts of each of these phospholipids can be present in equal amounts (i.e., 25% each by weight), or can be present in a range from about 5% to about 95% by weight each as long as the total does not exceed 100%.

The phospholipids can also comprise antioxidants, including tocopherol, BHT, BHA, TBQH, ethoxyquin, β-carotene, astaxanthin, fucoxanthin, canthaxanthin, vitamin E, and vitamin C to prevent degradation of the unsaturated fatty acid esters (e.g., omega-3s). The phospholipids and can further comprise binding agents, mono and triglycerides, including those derived from oils containing high-purity omega-3s or from concentrations of omega-3s, esters and ester compounds, and other thinning agents, including Sorbitol. This assists in making the phospholipids a flowable powder or liquid for further processing. Further, the phospholipids can be encapsulated using known encapsulation techniques or pressed into pills.

The therapeutic liposome composition can also include PEG or PEG compounds, such as Vitamin E TPGS. Optionally, the oral formulation of the therapeutic liposome composition can include an enteric coating to aid in the absorption of the therapeutic liposome by the intestine.

In solid oral formulations, the PEG components contain an average molecular weight from 150 to 9000, including 3015 to 4800, 1305 to 1595, 3600 to 4400, 4400 to 4800, 7000 to 9000, 6000 to 7500, and 3150 to 3685. The PEG is present in a ratio by weight percent phospholipid to PEG from 99:1 to 50:50, including 95:5 and 85:15. The PEG is a hard, opaque, granular solid under the following trade names: Carbowax® PEG 3350, Carbowax® PEG 1450, Carbowax® PEG 4000, Carbowax® PEG 4600, Carbowax® PEG 8000, and Carbowax® 6000. The PEG may also be any other commercial formulation suitable for food grade use.

In liquid oral formulations, the PEG components contain an average molecular weight from 190 to 630, including 190 to 210, 285 to 315, 380 to 420, and from 570 to 630. The PEG can be the following commercial products: Carbowax® PEG 200, Carbowax® PEG 300, Carbowax® PEG 400, and Carbowax® PEG 600.

The oral formulation of the therapeutic liposome composition can also contain additional compounds selected from the group consisting of Resveratrol, quinones, diferuloylmethanes, sterols, β-glucans, glucosamine, carotenoids, terpenes, xanthpohylls, omega-3 fatty acids, probiotics, prebiotics, and combinations thereof. This can be mixed with the PEG, PEG compounds, phospholipids, or lipid compositions.

In yet a further aspect, methods of treating various aliments and diseases with the disclosed therapeutic liposome compositions are disclosed by providing an effective amount of the therapeutic liposome composition, e.g., either orally or intravenously. Such aliments and diseases include, but are not limited to, Alzheimer's disease, inflammation (e.g., arthritis and Crohn's disease), cardiovascular disease (e.g., high cholesterol, heart disease, and hypertension), schizophrenia, stress, depression, cancer, ADD, AHD, ADHD, decreased libido, and menopause. The disclosed therapeutic liposome compositions can also modulate endogenous levels of lipoxins, resolvins, and protectins in humans and animals.

In yet further aspects, methods for making the therapeutic liposome compositions are disclosed. Such methods can include using ultrasonic and/or high pressure, which can be achieved by using a homogenizer or microfluidizer. The methods can also comprise solubilizing the phospholipids in a solvent, such as, but not limited to, organic solvent (e.g., alcohol, hexane, isohexane, chloroform, as well as various alcohol products used in food/pharma/nutraceutical processing); petrochemical products used in food/pharma/nutraceutical processing; polysorbates; sugar-alcohol compounds; Vitamin-E TPGS; PEGs, or combinations thereof. Typically, a solution of phospholipids in a solvent is suspended in a buffer solution containing the active compound under conditions sufficient to form therapeutic liposomes in which the phospholipids substantially encapsulate the active compound. The solvent can optionally be removed, e.g., by evaporation. Such methods can be accomplished either in an inert environment or under vacuum (excluding UHP techniques). Forming the therapeutic liposomes can also be done using sonication, homogenization, microfluidization, other UHP, laser, or high-shear mixing. Other suitable methods can be found in Morrissey, Lab Protocol for Preparing Phospholipid Vesicles (SUV) by Sonication, University of Illinois at Urbana-Champaign and Moore, Destruction of Liposomes in Water by Neutron Recoil, University of Chicago, Feb. 6, 2009, both of which are incorporated herein by reference in their entirety.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A therapeutic liposome composition comprising a therapeutic phospholipid liposome that comprises phospholipids having at least two fatty acid ester moieties each of which is independently C₁₆-C₂₂ fatty acid ester and an active compound that is encapsulated by said therapeutic phospholipid liposome.

2. The therapeutic liposome of claim 1, wherein said phospholipids are of the formula: 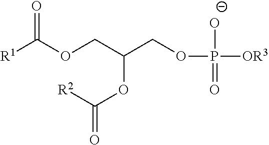
wherein
each of R¹ and R² together with the carboxylate group to which they are attached to is independently a C₁₆-C₂₂ fatty acid moiety; and
R³ together with the oxygen atom to which it is attached forms choline, serine, inositol, or ethanolamine moiety.

3. The therapeutic liposome of claim 1, wherein said C₁₆-C₂₂ fatty acid is an omega-3 fatty acid, an omega-6 fatty acid, or an omega-9 fatty acid.

4. The therapeutic liposome of claim 3, wherein said C₁₆-C₂₂ fatty acid comprises α-linolenic acid (ALA), docosahexaenoic acid (DHA), eicosatetraenoic acid (ETA), eicosapentaenoic acid (EPA), stearidonic acid (SDA), γ-linolenic acid (GLA), calendic acid (CLA), docosapentaenoic acid (DPA), or a combination thereof.

5. The therapeutic liposome of claim 1, wherein said C₁₆-C₂₂ fatty acid comprises GLA, SDA, DPA, DHA, EPA, ETA-3, or a combination thereof.

6. The therapeutic liposome of claim 1, wherein said active compound comprises a drug or a nutraceutical compound.

7. The therapeutic liposome of claim 6, wherein said drug comprises an antibiotic, a statin, a calcium channel blocker, an ACE inhibitor, an α-agonist, an α-blocker, a rennin blocker, dexmethylphenidate, guanfacine, methylphenidate, an anti-inflammatory compound, or a combination thereof.

8. The liposome of claim 6, wherein said nutraceutical compound comprises resveratrol, a quinone, diferuloylmethane, a sterol, a β-glucan, glucosamine, a carotenoid, a terpene, a xanthpohyll, an omega-3 fatty acid, a probiotic, a prebiotic, or a combination thereof.

9. A method for making a therapeutic liposome composition comprising a therapeutic phospholipid liposome that comprises phospholipids having at least two fatty acid ester moieties each of which is independently C₁₆-C₂₂ fatty acid ester and an active compound that is encapsulated by said therapeutic phospholipid liposome, said method comprising:
combining a solubilized phospholipid mixture with a buffer solution comprising an active compound, wherein the solubilized phospholipid mixture comprises a solubilized phospholipid in a solvent; and
forming a therapeutic liposome composition comprising a therapeutic phospholipid liposome with an encapsulated active compound.

10. The method of claim 9, wherein said step of forming the therapeutic liposome composition comprises sonication, homogenization, microfluidization, laser, high-shear mixing, or a combination thereof.

11. The method of claim 9 further comprising the step of removing at least a portion of the solvent from the therapeutic liposome composition.

12. The method of claim 9, wherein the solvent comprises hexane, isohexane, chloroform, polysorbate, an alcohol, a sugar-alcohol compound; Vitamin-E TPGS; polyethylene glycol, a petrochemical, or a combination thereof.

13. The method of claim 9, wherein the phospholipid is of the formula: 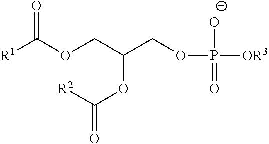
wherein
each of R¹ and R² together with the carboxylate group to which they are attached to is independently a C₁₆-C₂₂ fatty acid moiety; and
R³ together with the oxygen atom to which it is attached forms choline, serine, inositol, or ethanolamine moiety.

14. The method of claim 9, wherein the C₁₆-C₂₂ fatty acid is an omega-3 fatty acid, an omega-6 fatty acid, or an omega-9 fatty acid.

15. The method of claim 14, wherein the C₁₆-C₂₂ fatty acid comprises α-linolenic acid (ALA), docosahexaenoic acid (DHA), eicosatetraenoic acid (ETA), eicosapentaenoic acid (EPA), stearidonic acid (SDA), γ-linolenic acid (GLA), calendic acid (CLA), docosapentaenoic acid (DPA), or a combination thereof.

16. The method of claim 9, wherein the C₁₆-C₂₂ fatty acid comprises GLA, SDA, DPA, DHA, EPA, ETA-3, or a combination thereof.

17. The method of claim 9, wherein the active compound comprises a drug or a nutraceutical compound.

18. The method of claim 17, wherein the drug comprises an antibiotic, a statin, a calcium channel blocker, an ACE inhibitor, an α-agonist, an α-blocker, a rennin blocker, dexmethylphenidate, guanfacine, methylphenidate, an anti-inflammatory compound, or a combination thereof.

19. The method of claim 17, wherein said nutraceutical compound comprises resveratrol, a quinone, diferuloylmethane, a sterol, a β-glucan, glucosamine, a carotenoid, a terpene, a xanthpohyll, an omega-3 fatty acid, a probiotic, a prebiotic, or a combination thereof.
